(19) United States
(12) Patent Application Publication
(10) Pub. No.: US 2004/0038372 A1
(43) Pub. Date: Feb. 26, 2004

(54) Process for the production of amino acids with coryneform bacteria using phosphoglucose isomerases from coryneform bacteria

(75) Inventors: Brigitte Bathe, Salzkotten (DE); Achim Marx, Halle (DE); Walter Pfefferle, Halle (DE); Georg Thierbach, Halle (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/431,449

(22) Filed: May 8, 2003

Related U.S. Application Data
(60) Provisional application No. 60/379,391, filed on May 13, 2002.

(30) Foreign Application Priority Data
May 8, 2002 (DE) 102 20 574.4

(51) Int. Cl. C12N 9/92; C07H 21/04; C12P 21/02; C12N 5/06

(52) U.S. Cl. 435/234; 435/69.1; 435/320.1; 435/325; 536/23.2

(57) ABSTRACT

The invention relates to polynucleotide sequences of pgi genes encoding polypeptide sequences having phosphoglucose isomerase activity from coryneform bacteria, to coryneform bacteria containing such polynucleotides and/or polypeptides, a process for the production of L-amino acids using such polynucleotides and/or polypeptides, and methods of screening and amplifying polynucleotides encoding polypeptide sequences which comprise varying degrees of phosphoglucose isomerase activity.

### CROSS-REFERENCE TO RELATED APPLICATIONS

The present application claims priority to U.S. application Ser. No. 60/379,391, filed May 13, 2002. The entire content of this application is incorporated herein by reference.

### BACKGROUND OF INVENTION

1. Field of the Invention

The invention relates to polynucleotide sequences of pgi genes encoding polypeptide sequences having phosphoglucose isomerase activity from coryneform bacteria, to coryneform bacteria containing such polynucleotides and/or polypeptides, a process for the production of L-amino acids using such polynucleotides and/or polypeptides, and methods of screening and amplifying polynucleotides encoding polypeptide sequences which comprise varying degrees of phosphoglucose isomerase activity.

2. Discussion of the Background

L-amino acids, especially L-lysine, may be used in human medicine and in the pharmaceuticals industry, in the foodstuffs industry and in the feeding of animals.

Amino acids may be produced by fermentation of strains of coryneform bacteria, especially Corynebacterium glutamicum. Because of their great importance, attempts are continuously being made to improve the production processes. Improvements to the processes may include measures relating to the fermentation. Examples of such improvements include stirring and oxygen supply, or improvements in the composition of the nutrient media. Examples of such improvements to the nutrient media include the sugar concentration during the fermentation. Also, improvements may include improving the work up of the product form by ion-exchange chromatography. Finally, improvements may include those of the intrinsic performance properties of the microorganism itself.

In order to improve the performance properties of such microorganisms, methods of mutagenesis, selection and mutant selection may be employed. Such methods yield strains which may be resistant to antimetabolites. Examples of such antimetabolites include the lysine analogue S-(2-aminoethyl)-cysteine. Further, some methods yield auxotrophic strains for metabolites that are important in terms of regulation, and which produce L-amino acids.

For a number of years, methods of recombinant DNA technology have also been used for improving the strain of L-amino acid-producing strains of Corynebacterium glutamicum, by amplifying or attenuating individual amino acid biosynthesis genes and studying the effect on L-amino acid production.

The nucleotide sequence of the chromosome of Corynebacterium glutamicum belongs to the prior art and has been published, for example, within the scope of EP-A-1108790 and WO 01/00844.

Studies relating to the pgi gene and the enzyme phosphoglucose isomerase coded for by that gene are described in EP-A-1087015 and in WO 01/07626.

### SUMMARY OF THE INVENTION

An object of the present invention is to provide novel measures for improved preparation of L-amino acids or amino acids where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, including their salts (such as monohydrochloride or lysine sulfate).

One object of the present invention is a novel process for improving fermentative preparation of the L-amino acids, L-Lysine in particular. This process includes enhanced bacteria, preferably from Coryneform bacteria, which express attenuated amounts of phosphoglucose isomerase activity, which is encoded by the pgi gene.

Another object of the present invention is to provide such a bacterium, preferably from Coryneform bacteria, which expresses attenuated and/or enhanced pgi gene products. Another e object of the present invention is to provide such a bacterium, preferably from Coryneform bacteria, which expresses attenuated phosphoglucose isomerase activity.

Another object of the present invention is to provide a polynucleotide sequence encoding a polypeptide sequence with phosphoglucose isomerase activity. One embodiment of such a sequence is the polynucleotide sequence of SEQ ID NO. 1.

Another object of the present invention is a method of making phosphoglucose isomerase activity or a polypeptide having phosphoglucose isomerase activity. One embodiment of such a sequence is the polypeptide sequence of SEQ ID NO. 2.

Another object of the present invention relates to polynucleotide sequences encoding polypeptides having phosphoglucose isomerase activity and having the N-terminus optionally being shortened by 1 or 2, from 3 to 17, from 18 to 21, from 22 to 36 or from 37 to 46 amino acid residues, and nucleotide sequences that code for the mentioned proteins.

Another object of the present invention relates to polynucleotide sequences encoding polypeptides having phosphoglucose isomerase activity and having the N-terminus optionally being shortened by 1 or 2, from 3 to 17, from 18 to 21, from 22 to 36 or from 37 to 46 amino acid residues and the C-terminus having an amino acid sequence selected from the group SEQ ID NO:2 corresponding to position 47 to 585, SEQ ID NO:4 corresponding to position 47 to 585 and SEQ ID NO:6 corresponding to position 47 to 585, and nucleotide sequences that code for the mentioned proteins.

Another object of the present invention relates to polynucleotide sequences encoding polypeptides having phosphoglucose isomerase activity and having an amino acid sequence selected from the group SEQ ID NO:2, SEQ ID NO:2 corresponding to position 2 to 585, SEQ ID NO:4, SEQ ID NO:4 corresponding to position 2 to 585, SEQ ID NO:6 and SEQ ID NO:6 corresponding to position 2 to 585, and nucleotide sequences from coryneform bacteria that code for the mentioned proteins shown in SEQ ID NO:1, SEQ ID NO:3 and SEQ ID NO:5, including the variants arising from the degeneracy of the genetic code.

Other objects of the present invention include methods of detecting polynucleotides that are homologous to SEQ ID NO: 1 or those polynucleotides encoding polypeptides that have having phosphoglucose isomerase activity, methods of making such polynucleotides encoding such polypeptides, and methods of making such polypeptides.

The above descriptions highlight certain aspects and embodiments of the present invention. Additional objects, aspects, and embodiments of the present invention follow in the detailed description of the present invention considered together with the Figures.

### DETAILED DESCRIPTION OF THE INVENTION

Unless specifically defined, all technical and scientific terms used herein have the same meaning as commonly understood by a skilled artisan of molecular biology.

“Isolated” refers to a material, i.e. a polynucleotide separated out of its natural environment.

“Polynucleotide” in general relates to polyribonucleotides and polydeoxyribonucleotides, it being possible for these to be non-modified RNA or DNA or modified RNA or DNA. Polynucleotides, which comprise the sequences according to the invention, are furthermore suitable as primers, which code for phosphoglucose isomerase activity can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides which have a length of at least 31, 32, 33, 34, 35, 36, 37, 38, 39 or 40 or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

The expression “from coryneform bacteria” means that the corresponding proteins or nucleic acids come from coryneform bacteria or have their origin therein.

Nucleotide sequences that code for the proteins according to the invention having phosphoglucose isomerase activity can also be designated pgi genes or alleles.

The invention also provides bacteria, such as Corynebacterium glutamicum or Escherichia coli, which contain the nucleotide sequences, or pgi genes or alleles, according to the invention.

Likewise, vectors containing nucleotide sequences according to the invention are claimed.

The invention also provides coryneform bacteria in which proteins having phosphoglucose isomerase activity are present in enhanced or attenuated form, the mentioned proteins being characterised by a length of 585 amino acid residues, and the N-terminus of the mentioned proteins optionally being shortened by 1 or 2, from 3 to 17, from 18 to 21, from 22 to 36 or from 37 to 46 amino acid residues. The ranges for the number of amino acids shortened include all ranges and subranges, including 5, 7, 9, 11, 13, 15, 17, 19, 21, 23, 25, 27, 29, 31, 33, 35, 37, 39, 41, 43, and 45 nucleotides.

The term “enhancement” or “enhance” in this connection describes the increase in the intracellular activity or concentration of one or more enzymes or proteins in a microorganism that are coded for by the corresponding DNA, by, for example, increasing the number of copies of the gene or genes, using a strong promoter or using a gene or allele that codes for a corresponding enzyme or protein having a high level of activity, and optionally by combining those measures.

By the measures of enhancement, especially overexpression, the activity or concentration of the corresponding protein is generally increased by all ranges and subranges, including 10%, 25%, 50%, 75%, 100%, 150%, 200%, 300%, 400%, 500%, 1000%, and 2000%, based on that of the wild-type protein or the activity or concentration of the protein in the starting microorganism.

The term “attenuation” or “attenuate” in this context describes the diminution or exclusion of the intracellular activity or concentration of one or more enzymes or proteins in a microorganism that are coded for by the corresponding DNA, by, for example, using a weak promoter or using a gene or allele that codes for a corresponding enzyme having a low level of activity, or by inactivating the corresponding enzyme or protein or gene, and optionally by combining those measures.

By the measures of attenuation, the activity or concentration of the corresponding protein is generally lowered to from 0 to 75%, from 0 to 50%, from 0 to 25%, from 0 to 10% or from 0 to 5% of the activity or concentration of the wild-type protein, or of the activity or concentration of the protein in the starting microorganism. The ranges for the attenuation include all ranges and subranges, including 95, 90, 85, 80, 75, 70, 65, 60, 55, 50, 45, 40, 35, 30, 25, 20, 15, 10, and 5%.

Where L-amino acids or amino acids are mentioned hereinbelow, they are to be understood as being one or more amino acids. Examples of such amino acids, including their salts, may be selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine.

Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

Reference is made to standard textbooks of molecular biology that contain definitions and methods and means for carrying out basic scientific techniques, encompassed by the present invention. See, for example, Maniatis et al., Molecular Cloning: A Laboratory Manual, Cold Spring Harbor Laboratory, New York (1982) and Sambrook et al., Molecular Cloning: A Laboratory Manual, Cold Spring Harbor Laboratory, New York (1989) and various references cited therein.

Finally, the invention also provides a process for the production of one or more L-amino acids, especially selected from the group L-lysine, L-valine, L-threonine and L-methionine, with coryneform bacteria, which process is characterised by the following steps:

a) enhancement of proteins having phosphoglucose isomerase activity in the coryneform bacteria,

b) fermentation of the bacteria obtained in step a),

c) concentration of the amino acids in the medium, or in the fermentation liquor, or in the cells of the bacteria, and

d) isolation of the amino acids, constituents of the fermentation liquor and/or the biomass in totality or in part (from ≧0 to 100%) optionally remaining in the product,

the mentioned proteins being characterised by a length of 585 amino acid residues, and the N-terminus of the mentioned proteins optionally being shortened by 1 or 2, from 3 to 17, from 18 to 21, from 22 to 36 or from 37 to 46 amino acid residues.

Enhancement of the mentioned proteins having phosphoglucose isomerase activity is preferably used when the amino acid to be produced has a high NADPH consumption in the scope of its biosynthesis. That is the case, for example, with the amino acids L-lysine, L-valine, L-threonine and L-methionine.

The invention further provides a process for the production of one or more L-amino acids, especially selected from the group L-tryptophan, L-phenylalanine and L-tyrosine, with coryneform bacteria, which process is characterised by the following steps:

a) attenuation of proteins having phosphoglucose isomerase activity in the coryneform bacteria,

b) fermentation of the bacteria obtained in step a),

c) concentration of the amino acids in the medium, or in the fermentation liquor, or in the cells of the bacteria, and

d) isolation of the amino acids, constituents of the fermentation liquor and/or the biomass in totality or in part (from ≧0 to 100%) optionally remaining in the product,

the mentioned proteins being characterised by a length of 585 amino acid residues, and the N-terminus of the mentioned proteins optionally being shortened by 1 or 2, from 3 to 17, from 18 to 21, from 22 to 36 or from 37 to 46 amino acid residues.

Attenuation of the mentioned proteins having phosphoglucose isomerase activity is preferably used when a metabolite of the pentose phosphate cycle is a precursor of the amino acid to be produced. Erythrose-4-phosphate, for example, is a metabolite of the pentose phosphate cycle and a precursor of the aromatic amino acids L-tryptophan, L-phenylalanine and L-tyrosine. Summaries of the metabolic reactions and metabolites of the pentose phosphate cycle are to be found in textbooks of microbiology and biochemistry, such as, for example, the textbook of G. Gottschalk “Bacterial Metabolism” (2nd ed., Springer-Verlag, New York, USA, 1986).

The coryneform bacteria used may already produce L-amino acids before the enhancement or attenuation of the proteins according to the invention having phosphoglucose isomerase activity.

The microorganisms provided by the present invention may be able to produce L-amino acids from glucose, saccharose, lactose, fructose, maltose, molasses, starch, cellulose or from glycerol or ethanol or from acetic acid or lactic acid. They may be representatives of coryneform bacteria especially of the genus Corynebacterium. In the case of the genus Corynebacterium, special mention may be made of the species Corynebacterium glutamicum, which is known to those skilled in the art for its ability to produce L-amino acids.

Examples of suitable strains of the genus Corynebacterium, especially of the species Corynebacterium glutamicum, are especially the known wild-type strains

Corynebacterium glutamicum ATCC13032,

Corynebacterium acetoglutamicum ATCC 15806,

Corynebacterium acetoacidophilum ATCC 13870,

Corynebacterium melassecola ATCC 17965,

Corynebacterium thermoaminogenes FERM BP-1539,

Corynebacterium efficiens DSM44549

Brevibacterium flavum ATCC14067,

Brevibacterium lactofermentum ATCC13869 and

Brevibacterium divaricatum ATCC14020

and L-amino acid-producing mutants or strains prepared therefrom. Examples of such mutants or strains prepared therefrom are the L-lysine-producing strains

Corynebacterium glutamicum FERM-P 1709,

Brevibacterium flavum FERM-P 1708,

Brevibacterium lactofermentum FERM-P 1712,

Corynebacterium glutamicum FERM-P 6463,

Corynebacterium glutamicum FERM-P 6464,

Corynebacterium glutamicum DSM 5715,

Corynebacterium glutamicum DM58-1 and

Corynebacterium glutamicum DSM12866

and/or the the L-tryptophan-producing strains

Corynebacterium glutamicum ATCC21850 and

Corynebacterium glutamicum KY9218(pKW9901).

Strains designated “ATCC” can be obtained from the American Type Culture Collection (Manassas, Va., USA). Strains designated “FERM” can be obtained from the National Institute of Advanced Industrial Science and Technology (AIST Tsukuba Central 6, 1-1-1 Higashi, Tsukuba Ibaraki, Japan). Strains designated “DSM” can be obtained from the Deutsche Sammlung von Mikroorganismen und Zellkulturen (DSMZ, Braunschweig, Germany). The strain Corynebacterium glutamicum DM58-1 is described in EP-A-0358940. The strain Corynebacterium glutamicum KY9218 (pKW9901) is described in Ikeda et al. (Bioscience Biotechnology and Biochemistry 58 (4), 674-678 (1994)).

During work on the present invention, it was possible to identify proteins having phosphoglucose isomerase activity in Corynebacterium glutamicum, which are shown in SEQ ID NO:2, SEQ ID NO:4 and SEQ ID NO:6. N-terminal amino acids can be cleaved by enzymes proper to the host, so-called peptidases. A known enzyme is aminopeptidase, which cleaves N-terminal methionine.

For determining the N-terminal amino acid sequence of a protein such as phosphoglucose isomerase, the “His-tag” method can be used. In that method, the coding region of the corresponding gene is lengthened at the 3′ end generally by from 1 to 10, typically 6, histidine codons. To that end, the coding region is inserted into corresponding vectors, as are described in the prior art, for example in the Qiagen Product Guide 2002 from Qiagen GmbH (Hilden, Germany). It is also possible to prepare the corresponding gene by means of the polymerase chain reaction using oligonucleotide primers which contain the histidine codons, and then insert the gene lengthened by the histidine codons into a suitable vector. Expression of the protein preferably takes place in Escherichia coli or Corynebacterium glutamicum. The protein provided with histidine labelling is then purified from the crude extract by affinity chromatography and the N-terminus is determined by Edman degradation. Purification of the protein may also be carried out by two-dimensional gel chromatography.

It has also been found that an improvement in the lysine production of lysine-producing coryneform bacteria can be achieved when the proteins according to the invention having phosphoglucose isomerase activity are enhanced.

In order to achieve an enhancement, either the expression or the catalytic properties of the proteins according to the invention can be increased. The two measures are optionally combined.

In order to achieve an overexpression, the number of copies of the corresponding genes can be increased, or the promoter and regulation thregion or the ribosome binding site, which is located upstream of the structural gene, can be mutated. Expression cassettes inserted upstream of the structural gene have a similar effect. By means of inducible promoters it is additionally possible to increase the expression in the course of the production of amino acids by fermentation. Expression is also improved by measures to prolong the life of the m-RNA. Furthermore, the enzyme activity is also enhanced by preventing degradation of the enzyme protein. The genes or gene constructs may either be present in plasmids with different numbers of copies or be integrated and amplified in the chromosome. Alternatively, overexpression of the genes in question may also be achieved by changing the composition of the medium and the manner in which culturing is carried out.

One will find instructions thereon inter alia in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European patent specification 0 472 869, in U.S. Pat. No. 4,601,893, in Schwarzer and Puhler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in patent application WO 96/15246, in Malumbres et al. (Gene 134, 15-24 (1993)), in Jensen and Hammer (Biotechnology and Bioengineering 58, 191-195 (1998)), in Makrides (Microbiological Reviews 60:512-538 (1996)) and in known textbooks of genetics and molecular biology.

A common method of achieving an overexpression consists in the use of episomal plasmids. Suitable plasmids are those which are replicated in coryneform bacteria. Many known plasmid vectors, such as, for example, pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)), are based on the cryptic plasmids pHM1519, pBL1 or pGA1. Other plasmid vectors, such as, for example, those which are based on pCG4 (U.S. Pat. No. 4,489,160) or pNG2 (Serwold-Davis et al., FEMS Microbiology Letters 66, 119-124 (1990)) or pAG1 (U.S. Pat. No. 5,158,891), may likewise be used.

Suitable vectors may be those plasmid vectors with the aid of which the process of gene amplification by integration into the chromosome can be applied, as has been described by Reinscheid et al. (Applied and Environmental Microbiology 60, 126-132 (1994)) for the duplication or amplification of the hom-thrB operon. In the method according to Reinscheid et al., the complete gene is cloned into a plasmid vector that is able to replicate in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pGEM-T (Promega Corporation, Madison, Wis., USA), pCR2.1-TOPO (Shuman (1994). Journal of Biological Chemistry 269:32678-32684; U.S. Pat. No. 5,487,993), pCR®Blunt (Invitrogen, Groningen, Netherlands; Bernard et al., Journal of Molecular Biology, 234: 534-541 (1993)), pEM1 (Schrumpf et al., 1991, Journal of Bacteriology 173:4510-4516) or pBGS8 (Spratt et al., 1986, Gene 41: 337-342). The plasmid vector containing the gene to be amplified is then transferred to the desired strain of C. glutamicum by conjugation or transformation. The method of conjugation is described, for example, in Schäfer et al. (Applied and Environmental Microbiology 60, 756-759 (1994)). Methods of transformation are described, for example, in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over occurrence, the resulting strain contains at least two copies of the gene in question.

In order to achieve an attenuation, either the expression or the catalytic properties of the proteins according to the invention can be reduced. The two measures are optionally combined.

Gene expression can be diminished by carrying out the culturing in a suitable manner or by genetic alteration (mutation) of the signal structures of gene expression. Signal structures of gene expression are, for example, repressor genes, activator genes, operators, promoters, attenuators, ribosome-binding sites, the start codon and terminators. The person skilled in the art will find information thereon, for example, in patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)), and in known textbooks of genetics and molecular biology, such as, for example, the textbook of Knippers (“Molekulare Genetik”, 6th edition, Georg Thieme Verlag, Stuttgart, Germany, 1995) or that of Winnacker (“Gene und Klone”, VCH Verlagsgesellschaft, Weinheim, Germany, 1990). Methods of “anti-sense” RNA technology can also be used.

Mutations that lead to a change in or diminution of the catalytic properties of enzyme proteins are known from the prior art; examples which may be mentioned are the works of Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms”, Berichte des Forschungszentrums Jülichs, Jül-2906, ISSN09442952, Jülich, Germany, 1994). Summaries are to be found in known textbooks of genetics and molecular biology, such as, for example, that of Hagemann (“Allgemeine Genetik”, Gustav Fischer Verlag, Stuttgart, 1986).

Examples of mutations may be transitions, transversions, insertions and deletions of at least one base pair. Depending on the effect of the amino acid substitution on the enzyme activity, missense mutations or nonsense mutations may be included. As a result of nonsense mutations, sense codons are converted into stop codons and the translation breaks off prematurely. Insertions or deletions of at least one base pair in a gene lead to frame shift mutations, as a result of which incorrect amino acids are incorporated or the translation breaks off prematurely. Deletions of one or more codons typically lead to complete loss of enzyme activity.

The mentioned mutations are preferably incorporated into the nucleotide sequences of the genes which code for the N-terminus of the proteins according to the invention having phosphoglucose isomerase activity. The N-terminus includes especially the amino acid residues 1 to 22, 1 to 37 or 1 to 46, or 3 to 22, 3 to 37 or 3 to 46, or 18 to 22, 18 to 37 or 18 to 46, or 22 to 37, 22 to 46 or 37 to 46 of SEQ ID NO:2, 4 or 6. The ranges for the number of amino acids shortened include all ranges and subranges, including 5, 7, 9, 11, 13, 15, 17, 19, 21, 23, 25, 27, 29, 31, 33, 35, 37, 39, 41, 43, and 45 nucleotides.

The polynucleotides according to the invention include a polynucleotide encoding a polypeptide having phosphoglucose isomerase activity and SEQ ID NO:2, 4 or 6 or a fragment prepared therefrom and also those which are at least 70% to 80%, preferably at least 81% to 85%, particularly preferably at least 86% to 90%, and very particularly preferably at least 91%, 93%, 95%, 97% or 99% identical to the polynucleotide according to SEQ ID NO. 1 or a fragment prepared therefrom. The ranges for the percent identical include all ranges and subranges, including 72, 74, 76, 78, 80, 82, 84, 86, 88, 90, 92, 94, 96, and 98%.

The present invention provides polynucleotides which comprise the sequences according to the invention are suitable as hybridization probes for RNA, cDNA and DNA, in order to isolate, in the full length, nucleic acids or polynucleotides or genes which code for phosphoglucose isomerase activity ase or to isolate those nucleic acids or polynucleotides or genes which have a high similarity with the sequence of the pgi gene. They are also suitable for incorporation into so-called “arrays”, “micro arrays” or “DNA chips” in order to detect and to determine the corresponding polynucleotides.

Polynucleotides, which comprise the sequences according to the invention, are furthermore suitable as primers, which code for phosphoglucose isomerase activity can be prepared by the polymerase chain reaction (PCR).

Such oligonucleotides which serve as probes or primers comprise at least 25, 26, 27, 28, 29 or 30, preferably at least 20, 21, 22, 23 or 24, very particularly preferably at least 15, 16, 17, 18 or 19 successive nucleotides. Oligonucleotides which have a length of at least 31, 32, 33, 34, 35, 36, 37, 38, 39 or 40 or at least 41, 42, 43, 44, 45, 46, 47, 48, 49 or 50 nucleotides are also suitable. Oligonucleotides with a length of at least 100, 150, 200, 250 or 300 nucleotides are optionally also suitable.

The polypeptides according to the invention include a polypeptide according to SEQ ID NO:2, 4 or 6 or a fragment prepared therefrom, in particular those with the biological activity of phosphoglucose isomerase, and also those which are at least 70% to 80%, preferably at least 81% to 85%, particularly preferably at least 86% to 90%, and very particularly preferably at least 91%, 93%, 95%, 97% or 99% identical to the polypeptide according to SEQ ID NO. 2 and have the activity mentioned. The ranges for the percent identical include all ranges and subranges, including 72, 74, 76, 78, 80, 82, 84, 86, 88, 90, 92, 94, 96, and 98%.

Coding DNA sequences, which result from SEQ ID NO. 1 by the degeneracy of the genetic code, are also a constituent of the invention. In the same way, DNA sequences, which hybridize with SEQ ID NO. 1 or parts of SEQ ID NO. 1, are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the protein, i.e. are of neutral function. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences, which result in a corresponding manner from SEQ ID NO. 2, are also a constituent of the invention.

In the same way, DNA sequences, which hybridize with SEQ ID NO. 1 or parts of SEQ ID NO. 1, are a constituent of the invention. Finally, DNA sequences, which are prepared by the polymerase chain reaction (PCR) using primers, which result from SEQ ID NO. 1, are a constituent of the invention. Such oligonucleotides typically have a length of at least 15 nucleotides.

The skilled artisan will find instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides, which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

A skilled artisan will find instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

Instructions for the production of such mutations are part of the prior art and can be found in known textbooks of genetics and molecular biology, such as, for example, the textbook of Knippers (“Molekulare Genetik”, 6th edition, Georg Thieme Verlag, Stuttgart, Germany, 1995), that of Winnacker (“Gene und Klone”, VCH Verlagsgesellschaft, Weinheim, Germany, 1990) or that of Hagemann (“Allgemeine Genetik”, Gustav Fischer Verlag, Stuttgart, 1986).

The invention also provides nucleotide sequences that are substantially identical with the described nucleotide sequences. They include those nucleotide sequences which contain at least one further, especially conservative, amino acid substitution.

In the case of aromatic amino acids, the expression conservative substitution is used when phenylalanine, tryptophan and tyrosine are substituted for one another. In the case of hydrophobic amino acids, the expression conservative substitution is used when leucine, isoleucine and valine are substituted for one another. In the case of polar amino acids, the expression conservative substitution is used when glutamine and asparagine are substituted for one another. In the case of basic amino acids, the expression conservative substitution is used when arginine, lysine and histidine are substituted for one another. In the case of acid amino acids, the expression conservative substitution is used when aspartic acid and glutamic acid are substituted for one another. In the case of amino acids containing hydroxyl groups, the expression conservative substitution is used when serine and threonine are substituted for one another.

A common method of mutating genes of C. glutamicum is the method of gene disruption and gene replacement described by Schwarzer and Pühler (Bio/Technology 9, 84-87 (1991)).

In the method of gene disruption, a central portion of the coding region of the gene in question is cloned into a plasmid vector which is able to replicate in a host (typically E. coli), but not in C. glutamicum. Suitable vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-5465 (1992)), pGEM-T (Promega Corporation, Madison, Wis., USA), pCR2.1-TOPO (Shuman (1994). Journal of Biological Chemistry 269:32678-32684; U.S. Pat. No. 5,487,993), pCR®Blunt (Invitrogen, Groningen, Netherlands; Bernard et al., Journal of Molecular Biology, 234: 534-541 (1993)) or pEM1 (Schrumpf et al., 1991, Journal of Bacteriology 173:4510-4516). The plasmid vector containing the central portion of the coding region of the gene is then transferred to the desired strain of C. glutamicum by conjugation or transformation. The method of conjugation is described, for example, in Schäfer et al. (Applied and Environmental Microbiology 60, 756-759 (1994)). Methods of transformation are described, for example, in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over occurrence, the coding region of the gene in question is disrupted by the vector sequence, and two incomplete alleles lacking the 3′- and the 5′-end, respectively, are obtained. That method has been used, for example, by Fitzpatrick et al. (Applied Microbiology and Biotechnology 42, 575-580 (1994)) to exclude the recA gene of C. glutamicum.

In the gene replacement method, a mutation, such as, for example, a deletion, insertion or base substitution, is produced in vitro in the gene in question. The allele that is produced is in turn cloned into a vector that is not replicative for C. glutamicum, and the latter is then transferred to the desired host of C. glutamicum by transformation or conjugation. After homologous recombination by means of a first cross-over occurrence effecting integration and by means of a suitable second cross-over occurrence effecting an excision in the target gene or in the target sequence, incorporation of the mutation or of the allele is achieved. That method has been used, for example, by Peters-Wendisch et al. (Microbiology 144, 915-927 (1998)) to exclude the pyc gene of C. glutamicum by means of a deletion.

In addition, it may be advantageous for the production of L-amino acids, in addition to the enhancement or attenuation of the proteins according to the invention having phosphoglucose isomerase activity or of the genes or nucleotide sequences coding therefor, to enhance, especially overexpress, one or more enzymes of the biosynthesis pathway in question, of glycolysis, of the anaplerotic pathway, of the citric acid cycle, of the pentose phosphate cycle, of amino acid export, and optionally regulatory proteins.

The use of endogenous genes or endogenous nucleotide sequences is preferred. “Endogenous genes” or “endogenous nucleotide sequences” are to be understood as meaning the genes or alleles or the nucleotide sequences present in the population of a species.

Accordingly, for the production of L-lysine, it is possible, in addition to enhancing the proteins according to the invention having phosphoglucose isomerase activity, to enhance, especially overexpress, one or more genes selected from the group

the gene lysC coding for a feed-back resistant aspartate kinase (Accession No.P26512, EP-B-0387527; EP-A-0699759; WO 00/63388),

the gene dapA coding for dihydrodipicolinate synthase (EP-B 0 197 335),

the gene lysE coding for the lysine export protein (DE-A-195 48 222),

the gene pyc coding for pyruvate carboxylase (WO 99/18228, U.S. Pat. No. 6,171,833),

the gene gdh coding for glutamate dehydrogenase (U.S. Pat. No. 6,355,454),

the gene zwf coding for glucose 6-phosphate dehydrogenase (JP-A-09224661),

the gene mqo coding for malate:quinone oxidoreductase (Molenaar et al., European Journal of Biochemistry 254, 395-403 (1998)), and

the gene zwa1 coding for the Zwa1 protein (EP-A-1111062).

It can also be advantageous for the production of L-lysine, in addition to enhancing the proteins according to the invention having phosphoglucose isomerase activity, to attenuate, especially reduce or exclude the expression of, one or more genes selected from the group

the gene ccpA1 coding for catabolite control protein A (WO 02/18419),

the gene pck coding for phosphoenol pyruvate carboxykinase (EP-A-1094111),

the gene fda coding for fructose bisphosphate aldolase (Molecular Microbiology 3 (11), 1625-1637 (1989); ACCESSION Number X17313),

the gene zwa2 coding for the Zwa2 protein (EP-A-1106693).

The microorganisms produced according to the invention also form part of the invention and can be cultivated continuously or discontinuously by the batch process or by the fed batch or repeated fed batch process for the purposes of the production of L-amino acids. A summary of known cultivation methods is described in the textbook of Chmiel (Bioprozesstechnik 1. Einführung in die Bioverfahrenstechnik (Gustav Fischer Verlag, Stuttgart, 1991)) or in the textbook of Storhas (Bioreaktoren und periphere Einrichtungen (Vieweg Verlag, Braunschweig/Wiesbaden, 1994)).

The culture medium to be used must meet the requirements of the strains in question in a suitable manner. Descriptions of culture media for various microorganisms are to be found in the handbook “Manual of Methods for General Bacteriology” of the American Society for Bacteriology (Washington D.C., USA, 1981).

There may be used as the carbon source sugars and carbohydrates, such as, for example, glucose, saccharose, lactose, fructose, maltose, molasses, starch and cellulose, oils and fats, such as, for example, soybean oil, sunflower oil, groundnut oil and coconut oil, fatty acids, such as, for example, palmitic acid, stearic acid and linoleic acid, alcohols, such as, for example, glycerol and ethanol, and organic acids, such as, for example, acetic acid or lactic acid. Those substances may be used individually or in the form of a mixture.

There may be used as the nitrogen source organic nitrogen-containing compounds, such as peptones, yeast extract, meat extract, malt extract, corn steep liquor, soybean flour and urea, or inorganic compounds, such as ammonium sulfate, ammonium chloride, ammonium phosphate, ammonium carbonate and ammonium nitrate. The nitrogen sources may be used individually or in the form of a mixture.

There may be used as the phosphorus source phosphoric acid, potassium dihydrogen phosphate or dipotassium hydrogen phosphate or the corresponding sodium-containing salts. The culture medium must also contain salts of metals, such as, for example, magnesium sulfate or iron sulfate, which are necessary for growth. Finally, essential growth substances, such as amino acids and vitamins, may be used in addition to the above-mentioned substances. Suitable precursors may also be added to the culture medium. The mentioned substances may be added to the culture in the form of a single batch, or they may be fed in in a suitable manner during the cultivation.

In order to control the pH value of the culture, basic compounds, such as sodium hydroxide, potassium hydroxide, ammonia or ammonia water, or acid compounds, such as phosphoric acid or sulfuric acid, are expediently used. In order to control the development of foam, anti-foams, such as, for example, fatty acid polyglycol esters, may be used. In order to maintain the stability of plasmids, suitable substances having a selective action, such as, for example, antibiotics, may be added to the medium. In order to maintain aerobic conditions, oxygen or gas mixtures containing oxygen, such as, for example, air, are introduced into the culture. The temperature of the culture is normally from 20° C. to 45° C. and preferably from 25° C. to 40° C. The culture is continued until the maximum amount of the desired product has formed. That aim is normally achieved within a period of from 10 hours to 160 hours.

Methods of determining L-amino acids are known from the prior art. The analysis may be carried out as described in Spackman et al. (Analytical Chemistry, 30, (1958), 1190-1206) by anion-exchange chromatography with subsequent ninhydrin derivatisation, or it may be carried out by reversed phase HPLC, as described in Lindroth et al. (Analytical Chemistry (1979) 51: 1167-1174).

Numerous modifications and variations on the present invention are possible in light of the above teachings. It is, therefore, to be understood that within the scope of the accompanying claims, the invention may be practiced otherwise than as specifically described herein.

The present application claims priority to German Application No. DE 102 20 574.4, filed May 8, 2002. The entire content of this application is incorporated herein by reference.

What is claimed is:

1. An isolated polynucleotide sequence, which encodes a polypeptide having an amino acid sequence that is at least 70% identical to a polypeptide having an at least one amino acid sequence selected from the group consisting of SEQ ID NO. 2, SEQ ID NO. 4, and SEQ ID NO. 6, wherein the polypeptide has phosphoglucose isomerase activity.

2. The isolated polynucleotide sequence of claim 1, wherein the encoded polypeptide has an amino acid sequence that is at least 80% identical to a polypeptide having an at least one amino acid sequence selected from the group consisting of SEQ ID NO. 2, SEQ ID NO. 4, and SEQ ID NO. 6.

3. The isolated polynucleotide sequence of claim 1, wherein the encoded polypeptide has an amino acid sequence that is at least 90% identical to a polypeptide having an at least one amino acid sequence selected from the group consisting of SEQ ID NO. 2, SEQ ID NO. 4, and SEQ ID NO. 6.

4. An isolated polypeptide having an amino acid sequence that is at least 70% identical to a polypeptide having an at least one amino acid sequence selected from the group consisting of SEQ ID NO. 2, SEQ ID NO. 4, and SEQ ID NO. 6, wherein the polypeptide has phosphoglucose isomerase activity.

5. The isolated polynucleotide sequence of claim 4, wherein the polypeptide has an amino acid sequence that is at least 80% identical to a polypeptide having an at least one amino acid sequence selected from the group consisting of SEQ ID NO. 2, SEQ ID NO. 4, and SEQ ID NO. 6.

6. The isolated polynucleotide sequence of claim 4, wherein the polypeptide has an amino acid sequence that is at least 90% identical to a polypeptide having an at least one amino acid sequence selected from the group consisting of SEQ ID NO. 2, SEQ ID NO. 4, and SEQ ID NO. 6.

7. The isolated polypeptide according to claim 4, wherein the polypeptide has a length of 585 amino acids.

8. The isolated polypeptide according to claim 4, wherein the polypeptide has an N-terminus that is shortened by from 1 or 2, from 3 to 17, from 18 to 21, from 22 to 36 or from 37 to 46 amino acid residues.

9. The isolated polypeptide according to claim 4, wherein the polypeptide has an N-terminus that is shortened by from 1 or 2, from 3 to 17, from 18 to 21, from 22 to 36 or from 37 to 46 amino acid residues and the C-terminus having an amino acid sequence selected from the group consisting of amino acids 47 to 585 of SEQ ID NO:2, amino acids 47 to 585 of SEQ ID NO:4, and amino acids 47 to 585 of SEQ ID NO:6.

10. The isolated polypeptide according to claim 4, wherein the polypeptide has an amino acid sequence selected from the group consisting of SEQ ID NO:2, amino acids 2-585 of SEQ ID NO:2, SEQ ID NO:4, amino acids 2-585 of SEQ ID NO:4, SEQ ID NO:6, and amino acids 2-585 of SEQ ID NO:6.

11. A host cell, comprising the isololated polypeptide of claim 4.

12. The host cell of claim 11, which is a Corynebacterium glutamicum.

13. The host cell of claim 11, wherein the phosphoglucose isomerase activity is enhanced or attenuated.

14. A vector comprising the isolated polynucleotide of claim 1.

15. A host cell comprising the isolated polynucleotide sequence of claim 1.

16. The host cell of claim 14, which is a Corynebacterium glutamicum.

17. The host cell of claim 14, wherein the phosphoglucose isomerase activity are present in enhanced or attenuated form

18. A process for the production of amino acids, comprising
a) enhancing the production of the polypeptides according to claim 4 in a host cell,
b) fermenting the host cell,
c) concentrating the amino acids in a medium, or in the fermentation liquor, or in the host cell, and
d) isolating the amino acids, constituents of the fermentation liquor and/or biomass in totality or in part remaining in the product.

19. The process according to claim 18, wherin the host cell is Corynebacterium glutamicum.

20. The process according to claim 18, wherein the amino acids are one or more amino acids selected from the group consisting of L-lysine, L-valine, L-threonine, L-methionine, and mixtures thereof.

21. The process according to claim 18, characterised in that the amino acids are L-lysine.

22. A process for the production of amino acids, comprising
a) enhancing the production of the polypeptides according to claim 4 in a host cell,
b) fermenting the host cell,
c) concentrating the amino acids in a medium, or in the fermentation liquor, or in the host cell, and
d) isolating the amino acids, constituents of the fermentation liquor and/or biomass in totality or in part remaining in the product.

23. The process according to claim 22, wherein the host cell is Corynebacterium glutamicum.

24. The process according to claim 22, wherein the amino acids are one or more amino acids selected from the group consisting of L-tryptophan, L-phenylalanine, L-tyrosine, and mixtures thereof.


SEQUENCE LISTING (6 sequences)

SEQ ID NO: 1 - DNA, 1758 bp, Corynebacterium glutamicum
  CDS (1)..(1755)
atggccacgtcgaaaagcagcccaataaacgcacctaaatttgtcgtgtttcccactttgaacactcttcgatgcgcttggccacaaaagcaagctaacctgaagatgttatttaacgacaataaaggagttttcatggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggaatcaaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcatgccaggtaatcgcccaaccaccaccattttggcggaggaacttaccccttctattctcggtgcgttgatcgctttgtacgaacacatcgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtag

SEQ ID NO: 2 - protein, 585 aa, Corynebacterium glutamicum
MATSKSSPINAPKFVVFPTLNTLRCAWPQKQANLKMLFNDNKGVFMADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMESNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVMPGNRPTTTILAEELTPSILGALIALYEHIVMVQGVIWDINSFDQWGVELGKQQANDLAPAVSGEEDVDSGDSSTDSLIKWYRANR

SEQ ID NO: 3 - DNA, 1758 bp, Corynebacterium glutamicum
  CDS (1)..(1755)
atggccacgtcgaaaagcagcccaataaacgcacctaaatttgtcgtgtttcccactttgaacactcttcgatgcgcttggccacaaaagcaagctaacctgaagatgttatttaacgacaataaaggagttttcatggcggacatttcgaccacccaggcttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctcccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaacttttaccacccaggagacgctgtctaacgctcgtgcagctcgtgcttggctggtagagaagctcggtgaagaggctgtcgcgaagcatttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggaatcaaacggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcatgccaggtaatcgcccaaccaccaccattttggcggaggaacttaccccttctattctcggtgcgttgatcgctttgtacgaacacatcgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtag

SEQ ID NO: 4 - protein, 585 aa, Corynebacterium glutamicum
MATSKSSPINAPKFVVFPTLNTLRCAWPQKQANLKMLFNDNKGVFMADISTTQAWQDLTDHYSNFQATTLRELFKEENRAEKYTFSAAGLHVDLSKNLLDDATLTKLLALTEESGLRERIDAMFAGEHLNNTEDRAVLHTALRLPPEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMESNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVMPGNRPTTTILAEELTPSILGALIALYEHIVMVQGVIWDINSFDQWGVELGKQQANDLAPAVSGEEDVDSGDSSTDSLIKWYRANR

SEQ ID NO: 5 - DNA, 1758 bp, Corynebacterium glutamicum
  CDS (1)..(1755)
atggccacgtcgaaaagcagcccaataaacgcacctaaatttgtcgtgtttcccactttgaacactcttcgatgcgcttggccacaaaagcaagctaacctgaagatgttatttaacgacaataaaggagttttcatggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggagaccaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcgtgccaggtaatcgcccaaccaccaccattttggcggaggaacttaccccttctattctcggtgcgttgatcgctttgtacgaacacaccgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtag

SEQ ID NO: 6 - protein, 585 aa, Corynebacterium glutamicum
MATSKSSPINAPKFVVFPTLNTLRCAWPQKQANLKMLFNDNKGVFMADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMETNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVVPGNRPTTTILAEELTPSILGALIALYEHTVMVQGVIWDINSFDQWGVELGKQQANDLAPAVSGEEDVDSGDSSTDSLIKWYRANR